(19) United States
(12) Patent Application Publication
(10) Pub. No.: US 2025/0308706 A1
(43) Pub. Date: Oct. 2, 2025

(54) DEVELOPMENT OF A PERSONALISED PERIODONTITIS SCORE FOR PATIENT RISK STRATIFICATION AND TARGETED THERAPY

(71) Applicant: ParoX GmbH, Leipzig (DE)

(72) Inventors: Yann GAGER, Leipzig (DE); Pablo Sandro CARVALHO SANTOS, Leipzig (DE); Jörg GABERT, Leipzig (DE); Moritz KEBSCHULL, Birmingham (GB)

(21) Appl. No.: 19/235,429

(22) Filed: Jun. 11, 2025

Related U.S. Application Data
(63) Continuation of application No. 18/249,234, filed on Apr. 14, 2023.

(30) Foreign Application Priority Data
Oct. 14, 2020 (WO) PCT/EP2020/078896

(51) Int. Cl. G16H 50/30; C12Q 1/689

(52) U.S. Cl. CPC G16H 50/30; C12Q 1/689

(57) ABSTRACT

The present disclosure concerns the development of personalised periodontitis score to assess the individual level of microbial imbalances in periodontal pockets and/or saliva of a specific individual at a defined stage of treatment, relative to a predicted expected score for the average patient with similar systemic and local medical characteristics and demographic profile. The microbial profile obtained from genetic/genomic information is combined with clinical and demographical information in a mixed-effect model to obtain a predicted value and a standard error for a given set of clinical parameters. The comparison between this predicted average score and the individual patient personalised periodontitis score informs about the relative periodontal disease activity in this site and/or patient and thus allows for the stratification of the local site and/or the patient into risk categories and for the recommendation of targeted patient-specific treatment modalities.

### CROSS REFERENCE TO RELATED APPLICATIONS

The present application is a continuation of U.S. Non-Provisional patent application Ser. No. 18/249,234, entitled “DEVELOPMENT OF A PERSONALISED PERIODONTITIS SCORE FOR PATIENT RISK STRATIFICATION AND TARGETED THERAPY”, and filed on Apr. 14, 2023. U.S. Non-Provisional patent application Ser. No. 18/249,234 is a U.S. National Phase of International Application No. PCT/EP2021/078480 entitled “DEVELOPMENT OF A PERSONALISED PERIODONTITIS SCORE FOR PATIENT RISK STRATIFICATION AND TARGETED THERAPY,” and filed on Oct. 14, 2021. International Application No. PCT/EP2021/078480 claims priority to International Application No. PCT/EP2020/078896 filed on Oct. 14, 2020. The entire contents of each of the above-listed applications are hereby incorporated by reference for all purposes.

### TECHNICAL FIELD

The present disclosure concerns the development of personalised periodontitis score to assess the individual level of microbial imbalances in periodontal pockets and/or saliva of a specific individual at a defined stage of treatment, relative to a predicted expected score for the average patient with similar systemic and local medical characteristics and demographic profile. The microbial profile obtained from genetic/genomic information is combined with clinical and demographical information in a mixed-effect model to obtain a predicted value and a standard error for a given set of clinical parameters. The comparison between this predicted average score and the individual patient personalised periodontitis score informs about the relative periodontal disease activity in this site and/or patient and thus allows for the stratification of the local site and/or the patient into risk categories and for the recommendation of targeted patient-specific treatment modalities.

### BACKGROUND

Periodontitis is the most common non-communicable disease in humans, caused by a destructive interaction between a dysbiotic biofilm and a dysregulated host immune-inflammatory response. Periodontitis leads to tooth loss and is independently associated with pre-mature mortality. Oral diseases (mostly periodontitis) are globally responsible for more years lost to disability than any other human disease. Importantly, the detrimental effects—both in the oral cavity and systemically—are especially critical in patients with rapidly progressing variants of the disease.

Periodontitis is per se relatively easy and predictable to treat. The real challenge, however, is to detect/predict those patients with rapidly progressing disease before permanent damage occurs, and to thus identify patients that would benefit from a more intensive therapy. This is especially critical in individuals suffering from co-morbidities related to periodontitis and/or risk factors for periodontal disease. There are currently no diagnostic tools available to address these challenges.

Specifically, whilst substantial published evidence (and in part commercial products) exists pointing to single periodontal bacteria, groups/complexes of bacteria, or ecological measures of microbial balance/imbalance (see Table 1 for examples) as markers of disease versus health, and/or as markers of present disease severity, none of these diagnostic measures takes into account co-morbidities, local and systemic risk factors, or demographic information, allowing for stratification of sites or patients in relation to the relative risk. In summary, previously described molecular microbial measures of periodontitis can give information about the disease state, but do not reveal whether—for a given clinical situation
the level of microbial imbalance is similar to the average patient with similar characteristics, or substantially worse or better. Only this information would allow targeting personalised therapeutic approaches to individual patients and/or sites, reducing costly and harmful over-therapy whilst making sure the affected individuals obtain the interventions they require.

### DESCRIPTION OF THE INVENTION

The problem of the invention is solved by the method of claim 1, i.e., by a method for the patient- and site-specific assessment of microbial imbalances in periodontitis whilst accounting for critical clinical and demographic parameters, comprising the steps of
identification and quantification of bacterial species using genetic workflows in samples obtained from periodontal pockets or saliva;
calculation of a microbial profile based on the quantity of one or several bacterial species associated with periodontitis or a ratio of the quantities of these bacteria;
using a reference database, modelling of the microbial profile as a function of a selection of relevant clinical and demographical parameters;
using a set of clinical and demographical parameters from a patient, calculation of the predicted value of the microbial profile via the aforesaid model;
comparison of the microbial profile between the predicted value and the patient value obtained in the laboratory to assess the relative imbalance of the local microbiome and based on this, recommend a site and/or patient-specific therapy.

The present invention relies on the integrated analysis of several key points to allow for an innovative stratification and personalised targeted therapy of patients suffering from untreated or treated periodontitis, or that could be at risk of developing periodontitis.

The Personalised Periodontitis Score is dependent on many parameters, e.g. selected from:
the genetic/genomic method to measure microbial profiles
the selection of bacterial species assessed in the microbial profile
the local and systemic clinical, as well as demographic parameters for the patient or site (e.g., a sample from an 8 mm pocket from a molar in supportive periodontal therapy [local clinical factors] from a non-smoking, systemically healthy [systemic clinical factors] white Caucasian male of 51 years [demographic factors]), and
the data available in the reference database.

Biological samples to be used in the method are samples from periodontal pockets or saliva. All types of periodontal pockets can be sampled. This requires a dental professional. Saliva sampling is very straightforward and does not require substantial infrastructure or skilled personnel. There is no limitation to gender, age and origin, or stage of therapy.

Clinical information means all relevant medical information associated with each sample/patient. Relevant clinical parameters for the present disclosure include:
Local factors: pocket depth of the sampled site (continuous, in mm), bleeding-on-probing (dichotomous), tooth type (categories: anterior, premolar, molar), therapy stage (categories: untreated, in active therapy, in supportive therapy)
Systemic factors smoking status (categories: current, past, never) as well as diabetes status (categories: yes/controlled, yes/uncontrolled, no).

Demographic information is information about groups of people. Relevant demographic parameters for the present disclosure include:
age (continuous, in years),
gender (categories: male, female, transgender), and
(optionally) race and ethnicity.

Genetic/genomic workflows comprise all the steps from nucleic acid extraction to the quantification of the sample based on different genetic methods including DNA-DNA hybridisation, quantitative PCR and different Next-Generation Sequencing methods.

A microbial profile is the quantity of one bacterial species or the sum of several bacterial species identified as key markers of periodontitis or periodontal health, or, as an extension, ratios between the sums of selected species. A microbial profile can be calculated based on the number of any bacterial species, which are identified as key markers of periodontitis or periodontal health. Several bacterial species mean a selection of two to 700 bacterial species (i.e. the number of common phylotypes known in the human mouth). The number of bacterial species identified as key markers is at least one, preferably between 2 to 700, more preferably between 2 to 600, 2 to 500, 2 to 400, 2 to 300, 2 to 200 or 2 to 100. Most preferably the number of bacterial species identified as key markers is between 2 to 90, 2 to 80, 2 to 70, 2 to 60, 2 to 50, 2 to 40, 2 to 30, 2 to 20 or 2 to 10. In an especially preferred embodiment, the number of bacterial species identified as key markers is 1, 2, 3 or 4 and the bacterial species are those as listed in Table 1. The unit of the microbial profile is based on the genetic/genomic method used. As a matter of example, the unit of the microbial profile Next-Generation Sequencing (NGS) is NGS reads.

The reference database compiles the following information about multiple samples from different sites and patients:
Local and systemic clinical information, as outlined above
Demographical information, as outlined above
as well as microbial profile for a given genetic/genomic method and selection of bacterial species

This reference database is an essential prerequisite for the calculation of the Personalised Periodontitis Score.

A given combination of clinical and demographic information can be used to stratify the sites and/or patients from the reference data base. Specific stratifications are expected to have higher values—indicating higher levels of microbial imbalances and thus increased disease activity—of the Personalised Periodontitis Score because patients present one or several risk factors like age and smoking status that are well-established risk factors for the disease. A typical stratification for the reference database could be smoking males between 50 and 59 years.

A set of defined clinical and diagnostic parameters are required to calculate the average expected score and the Personalised Periodontitis Score for a given genetic/genomic method and selection of microbial profile. As a matter of example in this disclosure, we calculated the scores for non-smoking males of 51 years for three microbial profiles. The linear mixed-effect model for this selection of parameters on the reference database is used to calculate an average expected score as well as associated standard errors to this average. The linear mixed-effect model used in the method of the present invention is known to the person skilled in the art and is described Brown, 2021, the disclosure of which is incorporated herein in its entirety, especially in regard to the description of the linear mixed-effect model on pp. 1 to 19. The Personalised Periodontitis Score from the sampling site is calculated directly with the selected genetic method (quantity of one or several bacterial species} and compared to the average expected score and associated standard errors obtained with the modelling and the reference database.

A Personalised Periodontitis Score within −1 SE and 1 SE of the predicted average expected score represents the most typical range of values. In a preferred embodiment, decreased scores have a Personalised Periodontitis Score below −1 SE of the predicted value. In a further preferred embodiment, increased scores have a Personalised Periodontitis Score below −1 SE of the predicted value.

On the basis of the above, quantities of bacteria can be detected using diverse molecular methods including “checkerboard” DNA-DNA hybridization, quantitative PCR, 165 amplicon sequencing, DNA whole genome sequencing/metagenomics, and/or RNA meta-transcriptomic sequencing. The present invention relies on the detection of microbial profiles which are measures of absolute or relative quantities of one or several marker bacteria associated with periodontitis or periodontal health (Table 1). All types of microbial profiles can be used for the present invention. Sometimes, a Microbial Dysbiosis Index or a Subgingival Microbial Dysbiosis Index is used as microbial profile in the method of the invention. A suitable Microbial Dysbiosis Index is e.g. described in Gevers, Kugathasan et al., 2014, which is incorporated herein in its entirety. A suitable Subgingival Microbial Dysbiosis Index is e.g. described in Chen, Marsh et al., 2021, which is incorporated herein in its entirety. The term microbial profile used in the rest of the document refers interchangeably to one of the molecular indices of Table 1 or any other microbial profile possible.

A Microbial Dysbiosis Index relates to the abundance of disease-related microorganism in patient samples and is defined as the log of [total abundance of microorganism increased in periodontitis] over [total abundance of organisms decreased in periodontitis] for all samples. The Microbial Dysbiosis Index shows a strong positive correlation with clinical disease severity and negative correlation with species richness, demonstrating that a severe disease state manifests a strongly reduced species diversity in favor of a more extreme dysbiosis.

A Subgingival Microbial Dysbiosis Index is defined as the mean centered log-ratio abundance of periodontitis-associated species/genera subtracted by that of health-associated species/genera and can be calculated according to the following method/formula: mean CLR abundance of dysbiotic DS/DG-mean CLR abundance of normobiotic DS/DG. The Subgingival Microbial Dysbiosis Index shows a high reproducibility, a strong positive correlation with clinical disease severity and discriminates between periodontitis and health with high accuracy.

Table 1: Examples of microbial profiles. A microbial profile is a measure of the quantity of one or several bacterial species identified as key markers of periodontitis or periodontal health, or the ratios of selected species. All of these microbial profiles are in the public domain and this disclosure does not intend to make any claims towards any of these profiles. These measures are merely meant to illustrate the use of the invention, the Personalised Periodontitis Score that compares any microbial profile obtained from a specific patient sample using the aforementioned molecular technologies to the expected average score of patients with similar characteristics, aiming to identify those samples that deviate significantly from this average for altered therapy.

| Profile | | |
|---|---|---|
| Number | Name | Bacterial species and/or reference |
| #1 | — | Porphyromonas gingivalis |
| #2 | Red | Porphyromonas gingivalis + Tannerella |
| | complex | forsythia + Treponema denticola |
| #3 | Etiologic | Aggregatibacter actinomycetemcomitans + |
| | bacterial | Porphyromonas gingivalis + Tannerella |
| | complex | forsythia + Treponema denticola |
| #4 | Microbial | (Gevers, Kugathasan et al. 2014) |
| | Dysbiosis | |
| | Index | |
| #5 | Subgingival | (Chen, Marsh et al. 2021) |
| | Microbial | |
| | Dysbiosis | |
| | Index | |

On the basis of the above, the present invention relies on a combination of a microbial profile, such as one of the profiles given in Table 1, with local and systemic clinical, as well as demographic parameters about sites and/or patients. As outlined above, clinical information means all relevant medical information associated with each sample/patient. Relevant clinical parameters for the present disclosure include:
Local factors: pocket depth of the sampled site (continuous, in mm), bleeding-on-probing (dichotomous), tooth type (categories: anterior, premolar, molar), therapy stage (categories: untreated, in active therapy, in supportive therapy).
Systemic factors smoking status (categories: current, past, never) as well as diabetes status (categories: yes/controlled, yes/uncontrolled, no).

Demographic information is information about groups of people. Relevant demographic parameters for the present disclosure include:
age (continuous, in years),
gender (categories: male, female, transgender), and
(optionally) race and ethnicity.

A linear mixed-effect model is then used to calculate a Personalised Periodontitis Score for the individual microbial profile, taking into account the aforementioned clinical and demographic characteristics and possibly multiple samples per patient. Linear mixed-effect models are an extension of simple linear models to allow both fixed effects (e.g pocket depth which has a defined range) and random effects (e.g., multiple samples per subject). Simple linear models include a simple linear regression where a dependent variable Y can be modelled by a dependent variable X. The response variable in the mixed model is the Personalised Periodontitis Score for the individual patient or site. Clinical and demographic parameters represent the explanatory variables in the model. Pocket depth, gender, age, smoking status and diabetes status are fixed effects while patient identity is a random effect. In detail, the equation of the linear mixed-effect model can be expressed as follows:

Personalised Periodontitis Score Local clinical factors+Systemic clinical factors+Demographical factors+(1 I patient)

The parameters from the equation have the following units:
Local clinical factors: pocket depth of the sampled site (continuous, in mm), bleeding-on-probing (dichotomous), tooth type (categories: anterior, premolar, molar), therapy stage (categories: untreated, in active therapy, in supportive therapy)
Systemic clinical factors smoking status (categories: current, past, never) as well as diabetes status (categories: yes/controlled, yes/uncontrolled, no).
Demographic information is information about groups of people. Relevant demographic parameters for the present disclosure include: age (continuous, in years), gender (categories: male, female, transgender), and (optionally) race and ethnicity.
Patient-numerical identifier in the clinical database (e.g., insurance number)

On the basis of the above, the site- or patient-related Personalised Periodontitis Score obtained in the laboratory from a patient can be compared to an average expected score with the same clinical and demographic parameters of all patient samples previously. As a matter of example, the set of clinical parameters can be defined as follows e.g., pocket depth=5 mm, gender=male, age=51 years, smoking status=non-smoking and diabetes status=yes. The predictive value of the model based on determined clinical parameter represents a value corrected from many possible confounding factors. The model is also used to calculate+/−1 standard error (SE) around the predicted value as a measure of uncertainty. The predictive value as well as the standard error can be theoretically calculated for all combination of clinical parameters incorporated in the model.

For a given set of clinical parameters, the predicted expected average score and the standard error can be used as a reference to compare to the Personalised Periodontitis Score of a patient or site obtained in the laboratory.

The Personalised Periodontitis Score is a single value that can be measured repeatedly for the same sampling site/patient to determine and monitor the stage of the disease during all stages of treatment, including in primary and secondary prevention. In other words, the values of the score for different samples taken at the same site but at different times can be compared.

The Personalised Periodontitis Score has the advantage that it can be systematically calculated for periodontal pockets or saliva of patients based on different molecular methods. In other words, it is possible to calculate and compare Personalised Periodontitis Score for the same molecular method.

While the model is established, the predicted value and the standard error of the Personalised Periodontitis Score based on a set of clinical parameters are dynamic. These values vary when adding new entries to the utilised reference database. The predictions gain in certainty with time considering that an increase of data points is associated with a reduction of the variance.

A patient- and site-specific characterization (tooth or oral cavity) is performed by comparing the Personalised Periodontitis Score for a specific periodontal pocket or oral cavity (via multiple samples and/or saliva sampling) to the predicted score for a specific set of clinical parameters using the linear mixed-effect model. Patients with samples showing decreased or increased Personalised Periodontitis Scores show a microbial imbalance and will receive targeted treatment differing from the treatment schedule recommended for the patients with Personalised Periodontitis Scores similar to the average predicted score.

Targeted treatment includes regular treatment and supervision without recommendation of additional anti-microbial measures, intensive treatment/supervision including antiseptic use as well as targeted use of local or, for generalised disease, systemic antibiotics.

Specifically, a sample characterised by a Personalised Periodontitis Score increased by more than one standard errors versus the predicted expected average score for a given clinical and demographic situation will be considered to exhibit increased microbial imbalances.

Conversely, for a given set of clinical and demographic parameters, a score of the Personalised Periodontitis Score decreased more than 1 SE versus the predicted average expected score represents the part of the population with lower-than-average microbial imbalances, in other words a periodontal pocket showing a profile that is more symbiotic than the rest of the sampled population.

Lastly, for a given set of clinical and demographic parameters, a Personalised Periodontitis Score within −1 SE and 1 SE of the predicted average expected score represents the most typical range of values, in other words, a situation showing a profile that is of the population with average microbial balance, in other words a periodontal pocket showing an average profile for dysbiosis in the sampled population.

This approach delivers representative and generalisable insights into the site- and patient-specific levels of microbial balance and allows for targeted treatment recommendations, including personalised intensive approaches including guided localised or systemic adjunctive antiseptics or antibiotic s.

A microbial imbalance can be categorized as follows:

Personalised Periodontitis Score below −1 SE of the average expected score: Regular treatment and supervision without recommendation of additional anti-microbial measures

Personalised Periodontitis Score between −1 SE+1 SE: mild microbial imbalance (corresponds to an early dysbiosis stage), requires intensive treatment/supervision including antiseptic use

Personalised Periodontitis Score above +1 SE: severe microbial imbalance (corresponds to advanced dysbiosis stage), requires very intensive treatment/tightly scheduled supervision, including the targeted use of local or, for generalised disease, systemic antibiotics

In addition to absolute measures of the level of symbiosis/dysbiosis, as detailed above, a patient- and site-specific characterization is performed by comparing the Personalised Periodontitis Score for a specific periodontal pocket or oral cavity to the average expected score for this clinical severity based on the data in the reference database, whilst taking into account the aforementioned confounders (local and systemic clinical factors, demographical factors) using regressions. Specifically, a sample characterised by one standard errors higher or lower Personalised Periodontitis Score than the average expected score for a given disease severity will be considered to exhibit increased or strongly increased dysbiosis or symbiosis, respectively. This approach delivers representative and generalisable insights into the site- and patient-specific dysbiosis levels and allows for targeted treatment recommendations, including personalised intensive approaches including guided localised or systemic adjunctive antiseptics or antibiotics.

The method of invention, in particular the Personalised Periodontitis Score has the advantage that it can be used to determine the individual level of microbial imbalance in periodontal pockets and/or the oral cavity and to correctly stratify individuals and/or sites for targeted personalized treatment interventions. These can include various anti-infective and/or periodontal maintenance interventions by the dentists or the dental team to influence microbial imbalances where they exist, and thus to avoid over-treatment, but also to avoid unnecessary and irreversible loss of periodontal tissues.

The method of invention, in particular the Personalised Periodontitis Score can, in addition, also be used to estimate the host response, since even though it measures relative quantities of microbial profiles, these were shown to be influenced directly by disproportionate host responses.

The method of invention, in particular the Personalised Periodontitis Score has the advantage that it can be used for monitoring the evolution of the stage and the development of the microbial balance for given periodontal pockets or the entire oral cavity over time. A biological sample from one of multiple periodontal pockets or a saliva sample can be sampled for given time intervals to monitor the evolution of the microbial balance in the periodontal pocket or oral cavity. Suitable and preferred time intervals for taking samples are e.g. every 1 day, every 2 days, every 3 days, every 4 days, every 5 days, weekly, 2-weekly or 3-weekly or monthly up to 5 months.

The method of invention, in particular the Personalised Periodontitis Score has the advantage that it can be used to check and monitor the effectiveness of treatment to a given patient. After a given treatment, the Personalised Periodontitis Score should decrease as a consequence of an improvement of the microbial balance. In the case of similar or higher values of Personalised Periodontitis Score, the switch to another adapted antibiotic is a recommended strategy to improve the treatment success. The monitoring of the efficacy of a periodontitis treatment preferably comprises the analysis of samples after or during the treatment that are taken from a periodontal pocket in a predetermined time interval, such as every 1 day, every 2 days, every 3 days, every 4 days, every 5 days, weekly, 2-weekly or 3-weekly or monthly up to 5 months.

The method of invention, in particular the Personalised Periodontitis Score has the advantage that it can be used to compare results from different sources for example dental practices or research teams. The complexity of microbial communities in periodontal pockets is summarized into a single number (predicted value) and range (−1 SE to +1 SE) for a given method that is self-explanatory and does not reference to other samples or databases.

The method of invention, in particular the Personalised Periodontitis Score has the advantage that it can be used as a proxy to monitor other diseases. Indeed, periodontitis has been associated to a vast number of diseases including diabetes mellitus, cardiovascular diseases like coronary heart disease and stroke, chronic respiratory diseases and rheumatoid arthritis. Patients showing high values of the Personalised Periodontitis Score characterizing relative microbial imbalances in periodontal pockets or the oral cavity may present higher risks to all the diseases mentioned above.

The molecular methods for detecting bacteria in periodontal pockets are known. These methods consist of “checkerboard” DNA-DNA hybridisation, quantitative PCR and Next-Generation Sequencing including 16 S ribosomal RNA sequencing, metagenomics and metatranscriptomics, among others.

The invention further relates to a combination of microbial profiles, comprised of one or more bacterial species which are key markers of periodontitis and/or periodontal health. The present invention covers all other microbial profiles resulting from one or several key bacteria associated positively or negatively with periodontitis, including ecological measures of microbial imbalances, such as indices quantifying microbial dysbiosis.

The invention is now further illustrated in three working examples illustrated by three figures for the microbial profile which comprises of 1) Porphyromonas gingivalis, 2) the Red complex (Porphyromonas gingivalis+Tannerellaforsythia+Treponema denticola) and 3) the Etiologic bacterial complex (Aggregatibacter actinomycetemcomitans+Porphyromonas gingivalis+Tannerella forsythia+Treponema denticola).

### SHORT DESCRIPTION OF THE FIGURES

FIG. 1 shows for Porphyromonas gingivalis the predicted values for a non-smoking male of 51 years (represented as squares) based on the model mentioned above and calculations based on a quantitative PCR dataset associated with clinical data. As a matter of example, the standard error around the predicted values and a fictive patient value (represented as triangle) is represented for the pocket depth 4 mm. The fictive patient value being above +1 SE of the average expected score, the patient requires very intensive treatment/tightly scheduled supervision.

FIG. 2 shows for the Red complex the predicted values for a non-smoking male of 51 years (represented as squares) based on the model mentioned above and calculations based on a quantitative PCR dataset associated with clinical data. As a matter of example, the standard error around the predicted values and a fictive patient value (represented as triangle) is represented for the pocket depth 4 mm. The fictive patient value being above +1 SE of the average expected score, the patient requires very intensive treatment/tightly scheduled supervision.

FIG. 3 shows for the Etiologic bacterial complex the predicted values for a non-smoking male of 51 years (represented as squares) based on the model mentioned above and calculations based on a quantitative PCR dataset associated with clinical data. As a matter of example, the standard error around the predicted values and a fictive patient value (represented as triangle) is represented for the pocket depth 4 mm. The fictive patient value being above +1 SE of the average expected score, the patient requires very intensive treatment/tightly scheduled supervision.

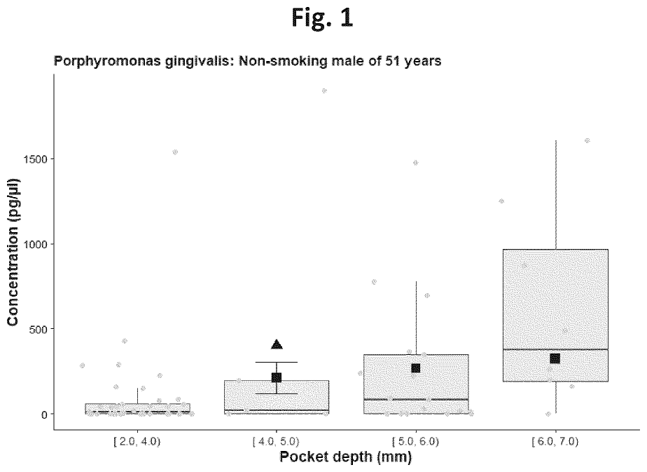

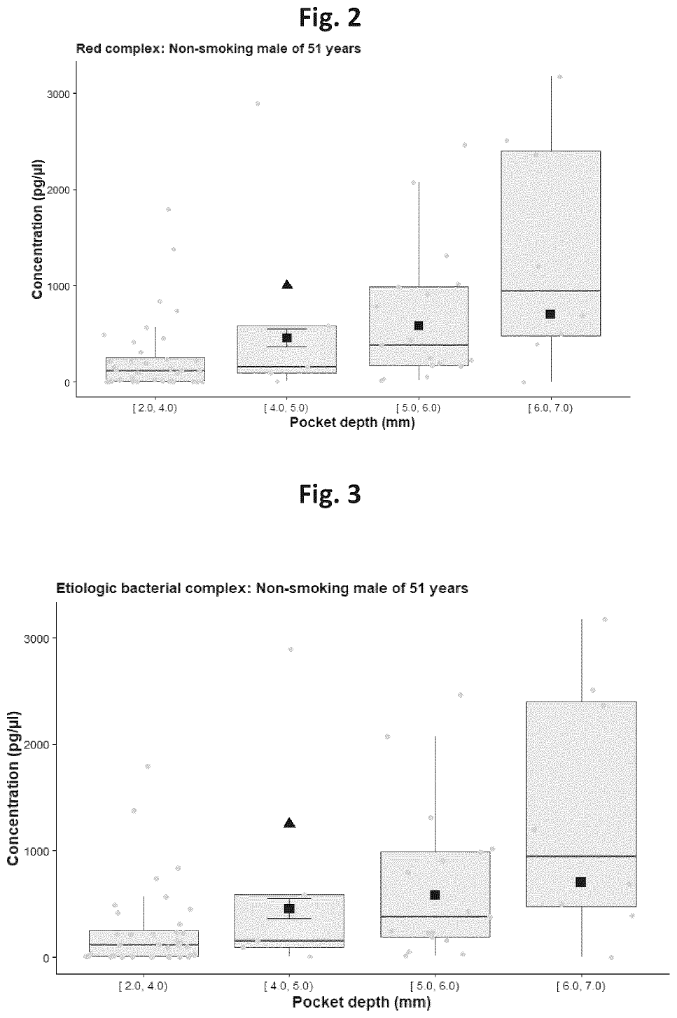

### EXAMPLE 1: PREDICTIVE MODELLING OF A MOLECULAR

### Personalised Periodontitis Score on Quantitative PCR Data

### Materials and Methods for the Calculation of the Microbial Profile and Modelling

The dataset comes from the supplementary material “DATA SHEET 51 I Dataframe and univariate analysis.” from the scientific paper:

Tomas I, Regueira-Iglesias A, Lopez M, Arias-Bujanda N, Novoa L, Balsa-Castro C and Tomas M (2017) Quantification by qPCR of Pathobionts in Chronic Periodontitis: Development of Predictive Models of Disease Severity at Site-Specific Level. Front. Microbial. 8:1443. doi: 10.3389/fmicb.2017.01443

The dataset consists of quantitative PCR (qPCR) information of different bacteria associated with periodontitis (columns “Concentration pg” in tab “qPCR data”). The dataset also contains clinical information (tab “Clinical Data”) including patient identity, age, gender, smoking status (in the four first columns) as well as pocket depth (both for control site in column “PPD_Control” and periodontitis site in column “PPD_Perio”). Information about the diabetes status was not available in the dataset and could therefore not be included in the analysis.

### Statistics

All analyses were done using the software R with custom-designed scripts. The clinical and qPCR information were matched thanks to the Patient ID. The following microbial profiles from Table 1 were calculated to illustrate the invention:
Profile #1 was the concentration of the bacterium Porphyromonas gingivalis in pg
Profile #2—Red Complex was calculated as the sum of the columns “Concentration pg” for the bacteria Porphyromonas gingivalis, Tannerel/aforsythia and Treponema denticola.
Profile #3-Etiologic bacterial burden was calculated as the sum of the columns “Concentration pg” for the bacteria Aggregatibacter actinomycetemcomitans+Porphyromonas gingivalis+Tannerel/aforsythia+Treponema denticola

The package lme4 from R was used to create the following linear mixed-effect model to obtain the average expected as well as associated standard errors to this average Personalised Periodontitis Score for a given category of parameters:

Imer(Microbial profile−Pocket depth+Gender+Age+Smoking status+(1I patient ID))

The model was then used to obtain a mean predicted value as well as the standard error for the different molecular profiles used for this illustration for a defined set of parameters: pocket depth between 4 and 10 mm, male as gender, 51 years as age and non-smoking as smoking status. The choice of the values from 4 to 10 mm pocket depth represents mild, moderate and severe cases, without those at risk to stem from periodontal-endodontic lesions with a different pathophysiology. The predicted value of the microbial profile for this set of clinical parameters was positively correlated with pocket depth for all used microbial indices, as expected.

The graphics can then be used to compare values for the three different microbial profiles. In this case we take the example of 400 pg/μI for profile #1, 1000 pg/μI for profile #2 and 1250 pg/μI for profile #for a pocket depth of 4 mm and the other clinical parameters comparable. For the three microbial profiles, the value is definitely above +1 SE of the average expected score. In these situations, the pocket would have a very dysbiotic profile which would

require a very intensive treatment possibly with antibiotics as well as tightly scheduled supervision.

### REFERENCES

Brown, V. A. (2021). “An Introduction to Linear Mixed-Effects Modeling in R.” Advances in Methods and Practices in Psychological Science 4(1): 1-19.
Chen, T., P. D. Marsh and N. N. AI-Hebshi (2021). “SMDI: An Index for Measuring Subgingival Microbial Dysbiosis.” J Dent Res: 220345211035775.
Gevers, D., S. Kugathasan, L. A. Denson, Y. Vazquez-Baeza, W. Van Treuren, B. Ren, E. Schwager, D. Knights, S. J. Song, M. Yassour, X. C. Morgan, A. D. Kostic, C. Luo, A. Gonzalez, D. McDonald, Y. Haberman, T. Walters, S. Baker, J. Rosh, M. Stephens, M. Heyman, J. Markowitz,
R. Baldassano, A. Griffiths, F. Sylvester, D. Mack, S. Kim, W. Crandall, J. Hyams, C. Huttenhower, R. Knight and R. J. Xavier (2 014). “The treatment-naive microbiome in new-onset Crohn's disease.” Cell Host Microbe 15 (3): 382-392.
Tomas I, Regueira-Iglesias A, Lopez M, Arias-Bujanda N, Novoa L, Balsa-Castro C and Tomas M (2017) Quantification by qPCR of Pathobionts in Chronic Periodontitis: Development of Predictive Models of Disease Severity at Sit e-Specifi c Level. Front. Microbial. 8:1443. doi: 10.3389/fmicb.2017.01443

1. A method for the patient- and site-specific assessment of microbial imbalances in periodontitis building on clinical and demographic parameters, comprising the steps of
identification and quantification of bacterial species using genetic workflows in samples obtained from periodontal pockets or saliva;
calculation of a microbial profile based on the quantity of one or several bacterial species associated with periodontitis or a ratio of the quantities of these bacteria;
using a reference database, modelling of the microbial profile as a function of a selection of relevant clinical and demographical parameters;
using a set of clinical and demographical parameters from a patient, calculation of the predicted value of the microbial profile via the aforesaid model;
comparison of the microbial profile between the predicted value and the patient value obtained in the laboratory to assess the relative imbalance of the local microbiome and based on this, recommend a site and/or patient-specific therapy,
wherein said clinical information includes
local factors: pocket depth of the sampled site, bleeding-on-probing, tooth type, therapy stage selected from untreated, in active therapy and in supportive therapy,
systemic factors smoking status as well as diabetes status,
said demographic information includes
age,
gender, and
optionally race and ethnicity,
and wherein for modelling of the microbial profile, a linear mixed-effect model is used, which is based on molecular and clinical data from a reference database, wherein the model includes the microbial profile as the response variable and clinical parameters including pocket depth as explanatory variables.

2. The method according to claim 1, wherein the microbial profile is defined as the quantity of one or several bacterial species identified as key markers of periodontitis, wherein several bacterial species mean a selection of two to 700 bacterial species, which represent the number of common phylotypes known in the human mouth.

3. The method according to claim 1, wherein the microbial profile is selected from ecological measures of microbial imbalances, such as indices quantifying microbial dysbiosis, specifically a Microbial Dysbiosis Index or a Subgingival Microbial Dysbiosis Index.

4. The method according to claim 1, wherein a linear mixed-effect model is based on molecular and clinical data from a reference database, wherein the model includes the microbial profile as the response variable and clinical parameters including pocket depth as explanatory variables and wherein the model can be used to predict the microbial profile as well as the standard error for any combination of parameters used in the model.

5. The method according to claim 1, wherein the microbial profile of an additional patient with defined clinical parameters can be compared to the predicted value and the standard error based on the exact same of clinical parameters.

6. The method according to claim 1, wherein a microbial imbalance stage is defined as follows:
Personalised Periodontitis Score <−1 SE from predicted value healthy stage
Personalised Periodontitis Score between −1/+1 SE from predicted value early dysbiosis stage
Personalised Periodontitis Score >+1 SE from predicted value advanced dysbiosis stage.

7. The method according to claim 1, wherein the bacterial species are identified by quantitative PCR, 16 S ribosomal RNA sequencing, shotgun sequencing or any other molecular techniques.

8. The method according to claim 1, wherein said method comprises determining the severity of periodontitis.

9. The method according to claim 1, wherein said method comprises the monitoring of dysbiosis development in periodontitis over time.

10. The method according to claim 1, wherein said method comprises—the monitoring of the efficacy of periodontitis treatment.

11. The method according to claim 1, wherein said method comprises the recommending and selecting a strategy for the treatment of periodontitis.

12. The method as claimed in 9, wherein said method comprises the analysis of samples that are taken from a periodontal pocket in a predetermined time interval, such as every 1 day, every 2 days, every 3 days, every 4 days, every 5 days, weekly, 2-weekly or 3-weekly or monthly up to 5 months.

13. The method as claimed in 10, wherein said method comprises the analysis of samples after or during the treatment that are taken from a periodontal pocket in a predetermined time interval, such as every 1 day, every 2 days, every 3 days, every 4 days, every 5 days, weekly, 2-weekly or 3-weekly or monthly up to 5 months.
